(19) United States
(12) Patent Application Publication
(10) Pub. No.: US 2015/0297755 A1
(43) Pub. Date: Oct. 22, 2015

(54) FLUORESCENCE COLORING FOR EYE SURGERY

(71) Applicant: CESACAR PARTICIPACIONS, S.L., Barcelona (ES)

(72) Inventor: Alain Philippe Telandro, le Cannet (FR)

(73) Assignee: CESACAR PARTICIPACIONS, S.L., Barcelona (ES)

(21) Appl. No.: 14/440,336

(22) PCT Filed: Nov. 1, 2013
(86) PCT No.: PCT/IB2013/003099

§ 371 Date: May 1, 2015

Related U.S. Application Data
(60) Provisional application No. 61/754,487, filed on Jan. 18, 2013.
(60) Provisional application No. 61/721,715, filed on Nov. 2, 2012.

(51) Int. Cl. A61K 49/00; A61B 19/00

(52) U.S. Cl. CPC A61K 49/0043; A61B 19/5202; A61K 49/0073; A61F 9/007

(57) ABSTRACT

Disclosed herein is a method of use of colored dye in ophthalmic surgery. In one embodiment the colored dye is fluorescent. In another embodiment the fluorescent dye is combined with viscoelastic gel for anterior segment eye surgery.

CROSS REFERENCE

This application claims the benefit of U.S. Ser. No. 61/721,715, filed Nov. 2, 2012; and U.S. Ser. No. 61/754,487, filed Jan. 18, 2013; the contents of both applications are incorporated by reference in their entirety.

### SUMMARY OF THE INVENTION

The present invention relates to formulations and methods incorporating coloration in ophthalmic surgical procedures.

In one embodiment, an ophthalmic solution comprising a therapeutically effective amount of a viscous or viscoelastic material and a coloring dye is provided during eye surgical procedure.

In another embodiment the viscoelastic material comprises a viscoelastic gel.

In one embodiment the coloration is a fluorescent viscoelastic gel.

In another embodiment a fluorophore is provided with viscoelastic gel with sodium hyaluronate structure.

In another embodiment a fluorophore is provided with viscoelastic gel with methylcellulose structure.

In one embodiment the fluorophore is fluorescein.

In one embodiment the ophthalmic solution is a fluorescent viscoelastic gel comprising hydroxypropylmethylcellulose and fluorescein.

In another embodiment the ophthalmic solution is a fluorescent gel comprising sodium hyaluronate and fluorescein.

In one embodiment the fluorescent viscoelastic gel is used in anterior segment eye surgery.

In one embodiment the viscoelastic gel and the fluorophore are formulated together.

In another embodiment the viscoelastic gel and the fluorophore are formulated separately.

In another embodiment the viscoelastic gel and the fluorophore are combined during surgery.

In another embodiment the colored viscoelastic gel is provided in phacoemulsification surgery such as cataract surgery.

In one embodiment the coloration is provided during infusion in phacoemulsification surgery.

In another embodiment the coloration is provided for irrigation of the anterior segment of the eye during phacoemulsification surgery.

In another embodiment several dyes will alternate during various step of phacoemulsification in particular for Hydro dissection lens nucleus.

In another embodiment each one of the dye is visualized with a suitable filter during the surgery.

In another embodiment fluorescent viscoelastic gel is provided in intraocular lens implant.

In another embodiment the fluorescent viscoelastic gel is provided during surgery for traumatic injury to the anterior segment of the eye.

In another embodiment the fluorescent viscoelastic gel is provided suring ciliary sclerotomy for the treatment of presbiopia.

In another embodiment the fluorescent viscoelastic gel is provided during glaucoma surgery.

One embodiment provides for illuminating the field of surgery with a portable microfibroscope providing monochromatic light, wherein the light of the microfibroscope is generated by fiber-optic.

In another embodiment the fiber-optic is incorporated in the surgery instrument.

In one embodiment the surgery instrument allows for anterior chamber eye surgery.

In another embodiment the monochromatic light is white.

In yet another embodiment the monochromatic light is blue.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

FIG. 1 illustrates the use of a microfibroscope for anterior chamber eye surgery.

FIG. 2 presents the use of colored infusion liquid (BSS) during cataract surgery.

FIG. 3 describes the use of micro eye endoscopy with the microfibroscope for aspiration of colored viscoelastic gel from the anterior chamber of the eye.

FIG. 4 demonstrates addition of fluorescent viscoelastic gel in a tubular mode, wherein the external coating is the fluorescent BSS.

FIGS. 5A and 5B illustrates the wave mode application of fluorescent viscoelastic gel.

### DETAILED DESCRIPTION OF THE INVENTION

There are a number of ophthalmic musculoskeletal and nerve surgical procedures performed by skilled surgeons which require or are facilitated by the use of a viscoelastic medium.

The anterior chamber of the eye is filled with a circulating liquid called aqueous humor or aqueous, whereas its posterior chamber is filled with vitreous humor or vitreous. The endothelial cell layer of the cornea is easily damaged and, once lost, these cells do not regenerate. The surgical procedures used in cataract surgery, corneal transplants and other types of ophthalmic surgery are likely to result in damage to these delicate cells unless measures are taken to protect them in the manner in which the aqueous does naturally.

In ophthalmic surgical procedures, except for non penetrating keratoplasty in which the corneal tissue is not fully penetrated, the recommended practice is to use an intraocular viscoelastic fluid for protecting the inner endothelial corneal surface and the delicate inner eye structures. Solutions that have been used in ophthalmologic surgical irrigation include normal saline, lactated Ringer's solution and Hartmann's lactated Ringer's solution, but these are not optimal due to potential unfavorable corneal and endothelial effects. Other aqueous solutions that include agents such as electrolytes, buffering agents for pH adjustment, glutathione and/or energy sources such as dextrose, better protect the tissues of the eye, but do not address other physiologic processes associated with surgery. One commonly used solution for ophthalmologic irrigation is a two part buffered electrolyte and glutathione solution disclosed in U.S. Pat. No. 4,550,022 to Garabedian et al., the disclosure of which is hereby expressly incorporated by reference. The two parts of this solution are mixed just prior to administration to ensure stability. These solutions are formulated with a goal of maintaining the health of ocular tissues during surgery.

Of the several substances that have been developed as substitutes for aqueous and vitreous, both as a protective layer covering the endothelial cells and as a coating on the surgical instruments and implanted material, sodium hyaluronate extracted from rooster combs, mixtures thereof or bioengineered forms of the naturally-occurring substance are widely employed. Once the surgical procedure is completed, the remaining vitreous/aqueous substitute is aspirated from the site using a syringe while remaining amounts are merely reabsorbed by the body in time without ill effects.

Methylcellulose has a long history of safe and effective use for ophthalmic applications. In 1945, Dr. Kenneth C. Swan studied the effects of methylcellulose on the ocular tissues of rabbit eyes. He suggested its use as a vehicle for ophthalmic drugs, to treat keratoconjunctivitis sicca and as an emollient. Then in 1959, Flemming, Merrill and Girard reported on further studies of methylcellulose in relation to irritation, hypersensitivity and its outflow from the anterior chamber of the rabbit eye.

The first reported use of methylcellulose as an intraocular lens coating serving to protect the corneal endothelium in rabbits was made by Drs. Kaufman and Katz in 1976. In the following year Dr. Paul Fechner reported upon the first human clinical use of methylcellulose to coat an intraocular lens prior to implantation.

Then in November of 1982, Dr. Danielle Aron-Rosa reported using methylcellulose in extracapsular surgery instead of high molecular weight sodium hyaluronate extracted from rooster combs which is very expensive. Shortly thereafter, Dr. Fechner amplified upon his earlier findings describing the use of methylcellulose as an intraocular viscous cushioning material in ophthalmic surgery.

The composition of the viscoelastic mixed gel slurries can vary within broad limits. The polymer solution in the mixture can constitute from 0.1 to 99.5%, preferably, from 0.5 to 99%, more preferably, from 1 to 95%, the rest being the gel phase. The choice of the proper composition of the mixture depends on the properties and composition of the two components and is governed by the desirable properties of the slurry and its final use.

The viscoelastic gel with varied density is used to protect the cornea by maintaining constant volume of the anterior chamber in place of the Aqueous humor. The surgical procedures using the phacoemulsification or small incision technique is performed in modern cataract surgery. Phacoemulsification surgery involves the use of a machine with microprocessor-controlled fluid dynamic. The phaco probe is a sophisticated microscopic, ultrasonic jack hammer which vibrates thousands at ultrasonic frequency pulverizes and liquidizes the cloudy cataract material. As the phacoemulsification probe is hollow, the debris created by this technique is aspirated through the tube of the phacoemulsification probe and led into a disposable chamber. Following complete removal of all the cataract material; the periphery of the capsular bag often has remnants left behind which are cleaned in an intervening stage called ‘I-A’ which stands for ‘irrigation-aspiration’ further viscoelastic gel is injected to the eye.

However, the disadvantage of using viscoelastic gel is the transparency of the gel and the difficulty to visualize the presence of the gel after surgery and the decrease of the transparency of the operative area; the use of a colored dye can alleviate these issues, however, such a dye should not decrease the transparency in the surgery; the color of the dye would allow for seeing the flows.

As disclosed herein, the combination of a dye and the monochromatic light would make it possible to intensify the visualization, for example the fluorescein diluted in the liquid infusion will be more visible on the blue light, filter that can easily be interposed at the source.

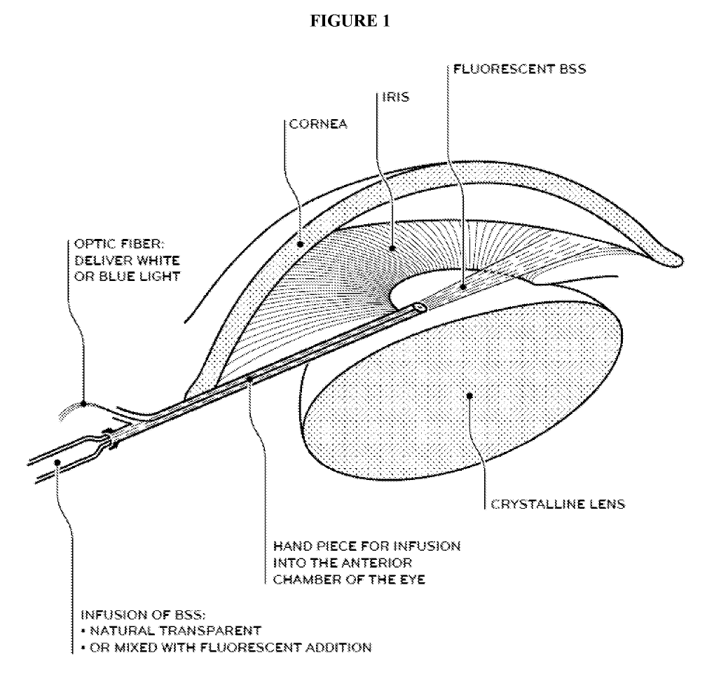

Lighting of the anterior chamber of the eye during surgery may depend on the microscope employed in the surgery and the retro lighting known as a pupillary gleam. To have effective lighting, the pupil needs to be dilated and the lighting center needs to be positioned in the visual center, which causes the phenomena of Purkinje. The system can be sophisticated comprising, for example, the addition of a fiber-optic connected to the infusion probe, this microfibroscope with either a white or blue light source illuminates with a tangential beam of light. FIG. 1 is a representation of the illumination field using the microfibroscope in the anterior chamber, allowing to clarify the flow of liquid from the inside of the probe.

The fiber-optic can also be added onto the probe of emulsification to view the suction of the liquid and the masses.

The variation in density of the dye also allows modulating the effect obtained with the dye into the anterior chamber helping to visualize the surgery site. A pulsed mode will allow for waves of dye, alternating clear phases and dense phases. The final washing of the anterior chamber will eliminate all of the dye. Several dyes can be alternatively used during surgery. A suitable filter downstream of the microscope will help to visualize the dye. The colorant will be biocompatible with the anterior segment of the eye with no toxicity.

A double electrical gallows controls the vials of the infusion liquid (BSS) which has a composition similar to that of aqueous humor and dyes independently: the height of each vial defines the density of the dye in the irrigation solution. The release of the alternate colors, lighting and fiber optic will be programed and controlled by solenoid valves allowing the irrigation solution in the anterior chamber of the eye. The visualization of the flow at the output of the infuser, its density, the laminar or turbulent aspect allows to better use the surgical tool and maximize the effectiveness during the surgery procedure.

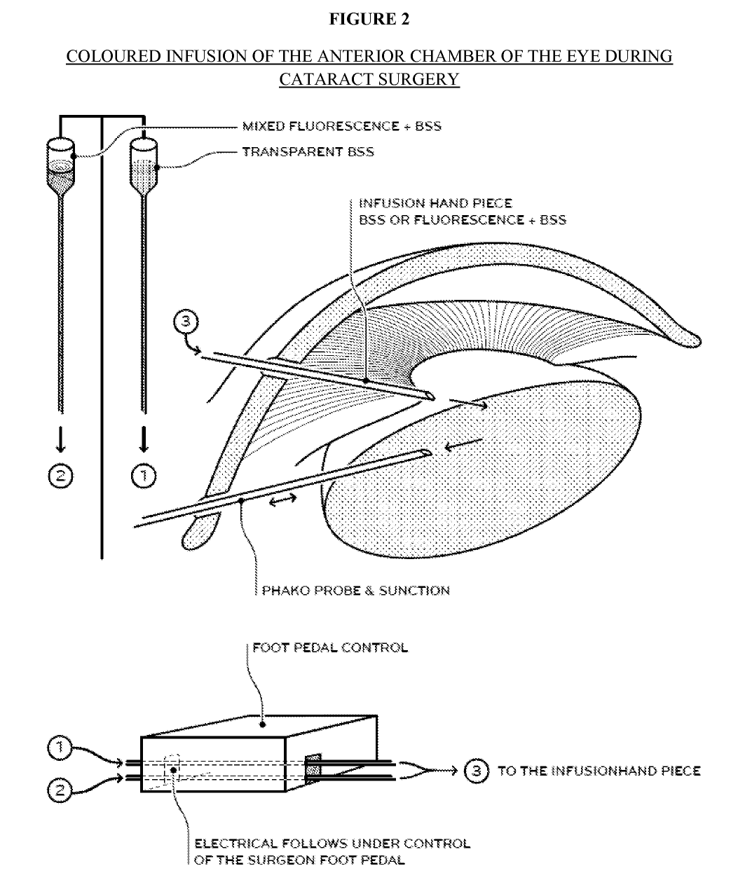

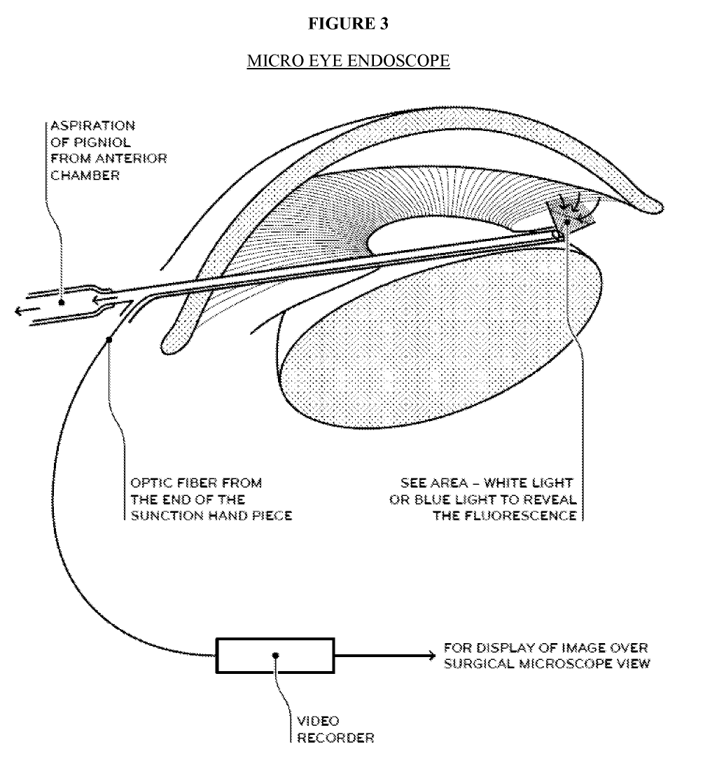

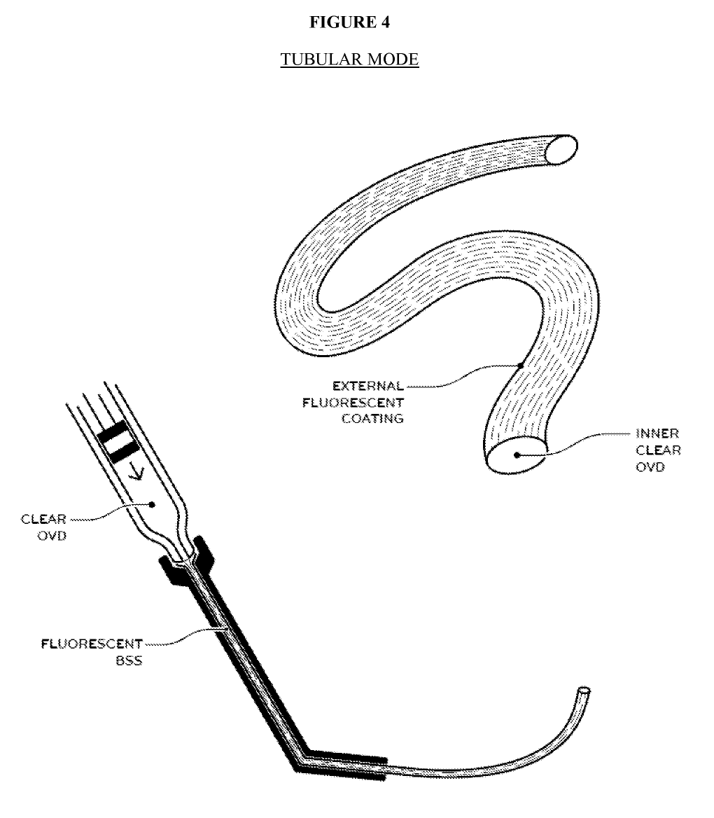

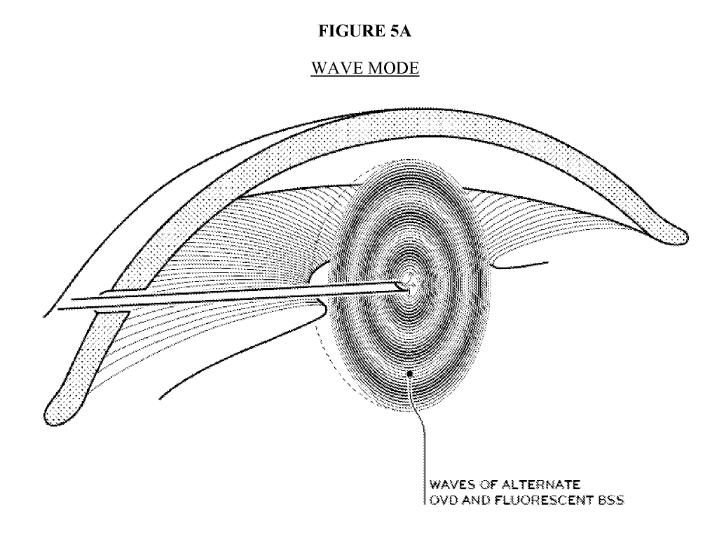

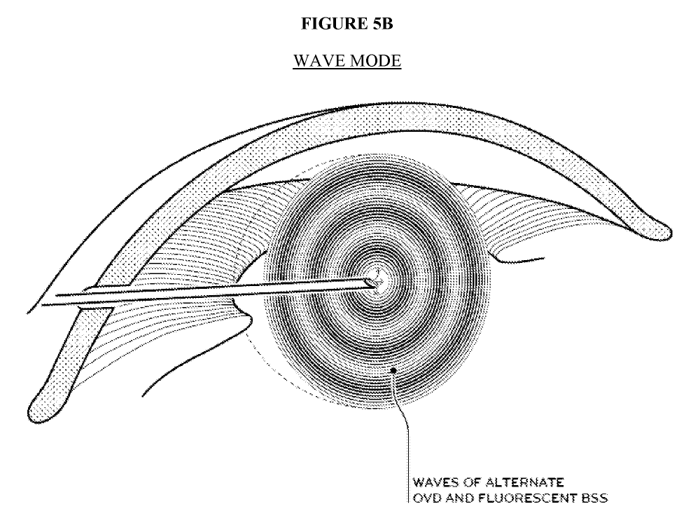

Illustrations of the methods disclosed herein are further illustrated by the appended fogures. FIG. 2, illustrates the use of a microfibroscope during cataract surgery, during the PHAKO infusion, emulsification or aspiration. FIG. 3 describes the use of micro eye endoscopy with the microfibroscope for aspiration of colored viscoelastic gel from the anterior chamber of the eye. The suction flow will also be viewed by its appearance, allowing to access any pre occlusion with crystalline or any reflux in case of occlusion.

The microfibroscope as an instrument with fiber-optics which conveys light may be advantageously employed in any type of surgery helping the surgeon avoid the zones of shades during the surgery.

The viscoelastic gel mixture according to the invention, contains a fluorophore in addition to the two major components namely, the polymeric gel slurry and the polymer solution.

One preferred fluorophore used with the viscoelastic gel is fluorescein, a synthetic organic compound. Fluorescein was first synthesized by Adolf von Baeyer in 1871; the sodium salt of fluorescein, is used extensively as a diagnostic tool in the field of ophthalmology and optometry, where topical fluorescein is used in the diagnosis of corneal abrasions, corneal ulcers and herpetic corneal infections. It is also used in rigid gas permeable contact lens fitting to evaluate the tear layer under the lens. It is available as sterile single-use sachets containing lint-free paper applicators soaked in fluorescein sodium. Intravenous or oral fluorescein is used in fluorescein angiography in research and to diagnose and categorize vascular disorders in e.g. legs, including retinal disease macular degeneration, diabetic retinopathy, inflammatory intraocular conditions, and intraocular tumors, and, increasingly, during surgery for brain tumors.

The fluorescence yield of the fluorescein molecule is very high and excitation occurs at 494 nm and emission at 521 nm for “Fluorescein sodium”; this allows for the detection of fluorescein at a very low concentration. The green fluorescence is detected under Ultraviolet light, using a blue emission filter (red-free).

The use of a fluorescent dye with commercially available viscoelastic gel will allow to visualize the smallest amount of the gel. In the case that the viscoelastic gel is colored, the presence of fluorescein will be easily detected using a blue emission filter placed downstream of the light source from the microscope. In operations such as iridio ciliary angle and space of implant of post capsule, where it is generally difficult to control and evaluate the presence of viscoelastic gel, the gel with fluorescein will reduce or eliminate such complications.

After using the fluorescent viscoelastic gel, the gel will be reduced or completely eliminated after surgery, and this in turn will decrease the risk for hypertonia, inflammation after surgery and accelerate visual recovery, resulting in overall less post operative stress for the patient.

For the surgeon the use of the fluorescein viscoelastic gel helps to visualize the liquid in the anterior chamber, which allows determination of the volume of the gel during different operational steps; aspiration of the gel at the end of the surgery; also allowing the use of ultraviolet light during the surgery by using a blue, red-free filter downstream of the microscope used for the surgery (example of a ZEISS microscope); reduction of surgery time; no major investment compared to transparent viscoelastic gel, by the addition of a red-free emission filter.

Various products are contemplated within the present disclosure which can be produced in different form to be used in different ophthalmologic treatments.

### EXAMPLES

### Example 1

Ampule containing the fluorescent viscoealstic gel reserved for intraocular lens implant to treat aphakia or other type of refractive disorders using a phakic ICL.

All type of viscoelastic gel can be used; dispersive; cohesive or joint. The name of each ampule can be clarifying the type of viscoelastic gel.
Visco D fluo: dispersive viscoelastic gel with fluorophore
Visco C fluo: cohesive viscoelastic gel with fluorophore
Visco M fluo: joint (mix) viscoelastic gel with fluorophore

The concentration of fluoresceine is at minimum and the fluorescence can just be seen in the presence of ultraviolet light.

The final formulation can also be as two separate ampoules; one ampule being the viscoalastic gel used for the first step of the surgery the second one is used for the IOL implant with the fluorescent dye.
BiviscoC/D fluo: dispersive viscoelastic gel with fluorophore
Bivisco D/C fluo: cohesive viscoelastic gel with fluorophore
BiviscoC/M fluo: joint (mix) viscoelastic gel with fluorophore
And so on

### Example 2

A gauge needle with fluorophore dye used with an ampule of standard viscoelastic gel. Having the needle with the fluorescein or Trypan blue allows the use of all commercial viscoelastic gels.

### Example 3

The walls of a catheter or cannula coated with a film of dry fluorescein that will dissolve with the viscoelastic gel during the injection.

### Example 4

An ampule of fluorescein diluted at some preferred concentration with viscoelastic gel.

1. A method of ophthalmic surgery comprising a step of administering to the eye a colored dye solution.

2. The method of claim 1 comprising injecting colored dye with viscoelastic gel.

3. A method of phacoemulsification surgery comprising a step of infusion with a colored dye aqueous.

4. A method of phacoemuslsification surgery comprising a step of irrigating with colored dye.

5. A method of phacoemulsification comprising alternating dyes administered in different phase of surgery.

6. The method of claim 1, wherein color visualization is obtained through a filter placed downstream of a light source of a microscope.

7. The method of claim 1, wherein said ophthalmic surgery is an anterior segment eye surgery.

8. The method of claim 1, wherein colored dye is fluorescent.

9. The method of claim 1, wherein the viscoelastic gel is fluorescent.

10. The method of claim 1, wherein the fluorophore with viscoelastic gel is fluorescein.

11. The method of claim 1, wherein the fluorescent viscoelastic gel is sodium hyaluronate.

12. The method of claim 1, wherein the fluorescent viscoleastic gel is methylcellulose.

13. The method of claim 1, wherein the fluorescent viscoelastic gel is used in phacoemulsification surgery such as cataract surgery.

14. The method of claim 1, wherein the fluorescent viscoelastic gel is used in intraocular lens implant.

15. The method of claim 1, wherein the fluorescent viscoelstic gel is used in traumatic injury of the anterior eye segment.

16. The method of claim 1, wherein the fluorescent viscoelstic gel is used in ciliary sclerotomy.

17. The method of claim 1, wherein the fluorescent viscoelstic gel is used in glaucoma surgery.

18. The method of claim 1, wherein viscoelastic gel and fluorophore are formulated together.

19. The method of claim 1, wherein viscoelastic gel and fluorophore are formulated separately.

20. The method of claim 1, wherein viscoelastic gel and fluorophore are combined during surgery.

21. An ophthalmic solution comprising a therapeutically effective amount of a viscous or viscoelastic material and a coloring dye.

22. The ophthalmic solution of claim 21, wherein the coloring dye comprises fluorescein.

23. The ophthalmic solution of claim 22, wherein the viscoelastic material comprises viscoelastic gel.

24. The ophthalmic solution of claims 23, wherein the viscoelastic gel comprises hydroxypropylmethylcellulose.

25. The ophthalmic solution of claims 24, wherein the viscoelastic gel comprises hydroxypropylmethylcellulose and fluorescein.

26. The ophthalmic solution of claims 23, wherein the viscoelastic gel comprises sodium hyaluronate.

27. The ophthalmic solution of claims 26, wherein the viscoelastic gel comprises sodium hyaluronate and fluorescein.

28. A method of surgery comprising: illuminating the field of surgery with a portable microfibroscope with monochromatic light generated by fiber-optic.

29. The method of claim 28, wherein the fiber-optic is incorporated in a surgery instrument.

30. The method of claim 28, wherein the surgery is an anterior chamber eye surgery.

31. The method of claim 28, wherein the monochromatic light is white.

32. The method of claim 28, wherein the monochromatic light is blue.
